(19) United States
(12) Patent Application Publication
(10) Pub. No.: US 2005/0048664 A1
(43) Pub. Date: Mar. 3, 2005

(54) Tracking Snow-A Liquid Solution mixture designed to aid in the visual location of trace amounts of game animal blood

(75) Inventor: Dixon Wayne Thayer SR., Frederic, WI (US)

Correspondence Address: ROBERT THAYER, 14337 PARK RD. LOT #4, GRANTSBURG, WI, 54840, US

(73) Assignee: Thayer, Frederic (US)

(21) Appl. No.: 10/604,930

(22) Filed: Aug. 27, 2003

(51) Int. Cl. G01N 33/00

(52) U.S. Cl. 436/166

(57) ABSTRACT

In many instances blood trails are lost due to the inability of the hunter to locate the sometimes, “invisible” blood spots. This invention will give the hunter a tool that will without question increase the odds of locating this wounded game animal. This invention will allow for a greater recognition of not only trace amounts of spotting blood, but also by locating changes in the animals movements and direction. Locating this blood spot will allow the hunter to continue his or her quest to find the wounded game animal. This invention clearly can reduce the number of game animals that could not be located in the wild, simply because cause of misread or overlooked blood spots.

### DETAILED DESCRIPTION

This invention is a Liquid Solution mixture. This Liquid Solution is comprised of Hydrogen Peroxide at a 4% by Volume mixture. This 4% by Volume solution is produced by mixing a proportionate amount of Conditioned Water to a Hydrogen Peroxide Catalyst of 34%. This quantitative 4% by Volume Liquid Solution is the required mixture to insure the desired results for its intended use. A Liquid Solution that is readily atomized in a typical “Spray Bottle” format.

Once in this format it is sprayed in an area known or assumed to have blood from a wounded game animal. In such events there are situations that seeing small trace amounts of game animal blood are misread. And in some instances overlooked completely, simply because of the size of the spot or because of the similarity of color in this natural setting. The above invention is designed to aid in the visual location of these trace amounts of game animal blood.

1. The above invention because of its natural reaction to game animal blood, will aid in the visual identification of hidden or misread signs from a wounded game animal.

2. The reaction of 4% hydrogen peroxide solution will transform the original blood spot to a “White” snow like substance. In addition this reaction increases the size of the original blood spot up to 3 to 4 times its original size. (The size of the blood spot after application will be dependent on its freshness)

3.

4. In any event it will be clearly larger than the original blood spot and because of its “white” appearance, will be clearly disassociated from the typical green to brown and in some cases red-ish appearing environment. Thus elevating the odds of finding a wounded game animal that is leaving these nearly invisible and undistinguishable trace amounts of spotting blood.
